(19) United States
(12) Patent Application Publication
(10) Pub. No.: US 2013/0056367 A1
(43) Pub. Date: Mar. 7, 2013

(54) Integrated sensing device and related methods

(71) Applicants: William E. Martinez, Berkeley, CA (US); Matthew R. Leyden, Berkeley, CA (US)

(72) Inventors: William E. Martinez, Berkeley, CA (US); Matthew R. Leyden, Berkeley, CA (US)

(21) Appl. No.: 13/573,257

(22) Filed: Sep. 4, 2012

Related U.S. Application Data
(60) Provisional application No. 61/573,465, filed on Sep. 6, 2011.
(60) Provisional application No. 61/634,907, filed on Mar. 8, 2012.

(51) Int. Cl. G01N 27/416; G01N 27/414; G01N 27/403; B82Y 5/00

(52) U.S. Cl. 205/792; 204/400; 204/403.01; 204/406; 205/775; 977/904

(57) ABSTRACT

The present invention is generally directed to devices and methods for sensing a variety of biologically-related substances and/or chemical substances. In a device aspect, the present invention is directed to a multilayer device for sensing metal ions, non-biological molecules, biological molecules, or whole cells. In a method aspect, the present invention is directed to a method for sensing species such as ions, protons, metal ions, non-biological molecules, whole cells, and biological molecules, for example one or more biologically-related substances such as proteins, nucleic acids, DNA, RNA, enzymes, and chemical substances such as water contaminants.

This application claims priority under 35 U.S.C. §119(e) to U.S. provisional patent application Ser. No. 61/573,465, filed Sep. 6, 2011, and to U.S. provisional patent application Ser. No. 61/634,907, filed Mar. 8, 2012, each of which is hereby incorporated by reference.

### FIELD OF THE INVENTION

The present invention is generally directed to devices and methods for sensing a variety of biologically-related and chemical substances in gas and fluid samples. It can also be used to detect and measure molecular interactions.

### BACKGROUND OF THE INVENTION

There is a growing need for reliable yet low cost early disease screening technologies, particularly in the area of cancers, where physicians and oncologist could be enabled to perform detection of cancers at early stages when the diseases are most treatable and treatments offer better survival rates for patients. Equally necessary is having technologies that can monitor, quantify, and analyze validated disease biomarkers so that physicians can reliably measure the physiological response of every patient to his/her personalized therapeutic treatment. This is perhaps the most significant challenge that needs to be met in order to move towards personalized medicine in the oncology arena. This is particularly important in the case of lung cancer because it is the leading cause of cancer-related deaths in Western nations and there are not existent molecular screening and early diagnostic tools.

In the US, lung cancer accounted for 15% of the new diagnosed cases and 28% of the deaths in 2010 (ACS Facts & Figures, 2010). These staggering figures call for major technological innovations to tackle these challenges. We believe Nanotechnology Biomachines (d.b.a. NanoTech Biomachines) has an important role to play here. The global market for in-vitro diagnostics (IVD) systems for cancer diagnostics reached US$ 3.8 billion in 2009, and its point-of-care IVD sector is growing by about 30% CAGR (Yole Development, 2010).

In its 2007 report, the National Institute of Health (NIH) provided estimates for the growing costs and expenditures related to battling cancer: direct medical costs and health expenditures ($89.0 billion); indirect morbidity costs due to lost productivity and illness ($18.2 billion); and, indirect mortality costs due to productivity loss and premature death ($112.0 billion).

One barrier to reducing the staggering number of cancer-related deaths and resulting health care costs is the lack of accurate, reliable and low cost early detection methods. The emerging field of precise molecular diagnostics provides windows of opportunity for the early detection of cancers, among other diseases, because it can enable the detection of molecular biomarkers and biological analytes at very small concentrations (nM, pM, and even fM). Emerging molecular diagnostic technologies provide opportunities for early cancer detection, as they can enable the detection of minute quantities of biomarker arrays. Current methods, however, are costly and time intensive: they require extensive sample preparation, complex hardware, sophisticated instrumentation and hours to days of analysis.

### SUMMARY OF THE INVENTION

The present invention is generally directed to devices and methods for sensing a variety of biologically-related and chemical substances in gas and fluid samples. The present invention can be used to measure absolute and relative concentrations of analytes (e.g. molecular species) in gas or fluid as well as measure label-free molecular interactions.

The present invention addresses the need for rapid, accurate, reliable and low cost ultra-sensitive detection and quantification of biological analytes . It can detect analytes at very low concentrations in gases and fluids, including the sensing of a variety of biologically-related and chemical substances, thereby facilitating the detection and screening of diseases. The present invention can also be used in the detection of biological species for national security applications. Other potential applications include the detection of metals, pollutants, biologically-related species in ground water, sea water and other water sources (environmental monitoring and remediation).

In a device aspect, the present invention is directed to a multilayer device for sensing metal ions, chemical substances, biological molecules, or whole cells. The device comprises: a) one or more cavities that provide for the introduction of a sample to be analyzed and one or more channels that provide for exit of the sample, or one or more channels that provide for the introduction and exit of the sample; b) one or more reservoirs that provide for the separation of different substances in a sample; c) one or more pillars that provide mechanical filtration of substances in a sample; d) one or more arrays of one or a plurality of nanostructures presented to the one or more cavities or one or more channels; e) a plurality of discrete electrical connectors and electrodes electrically connected to the one or more arrays of one or a plurality of nanostructures; and, (f) a reference gate electrode presented to the one or more cavities or one or more channels.

In a method aspect, the present invention is directed to a method for sensing species such as a metal, chemical substances, biological cells, and one or more types of biological molecules such as proteins and nucleic acids. The method comprises the steps of: a) introducing a solution of high affinity and selective binding elements into a device discussed above in the Summary of Invention Section, wherein the high affinity and selective binding species add functionality to the one or more arrays of one or more nanostructures by binding analyte and target species of interest to the surface of these nanostructures; b) introducing a buffer-electrolyte solution into one or more cavities, or one or more channels of the device, thereby allowing activation of arrays of one or more nanostructured-array field-effect-transistors (NSA-FETs) in the device for calibration and for setting a baseline current or voltage reference state; c) introducing a sample into the one or more cavities, or one or more channels of the device and determining any changes in the current or voltage (I/V) state of one or more nanostructured-array field-effect-transistors relative to their baseline state. The I/V changes can be used to measure the number of binding events of one or more analyte and target species of interest in the sample to high affinity and selectivity binding species on the surface of one or more nanostructures. These measured I/V changes can be used to quantify the concentration of analyte and target species of interest in a particular sample.

### BRIEF DESCRIPTION OF THE FIGURES

FIG. 1 shows a three-dimensional perspective sketch for the main components of one of the layers of a sensor according to the present invention.

FIGS. 2-8 show side view cross-sections of multiple different embodiments according to the present invention.

FIG. 9 shows a three-dimensional perspective sketch for the main components of one of the layers of a sensor according to the present invention.

FIGS. 10-19 show side view cross-sections of multiple different embodiments according to the present invention.

FIG. 20 shows a graph related to characterization of a surface functionalization of a graphene nanosheet sensor.

FIG. 21 shows a graph related to characterization of changes in surface potential as function of reference gate electrode potential.

FIG. 22 shows an illustration of a graphene nanosheet integrated sensor.

FIG. 23-24 shows illustrations of graphene nanomesh integrated sensors.

### DETAILED DESCRIPTION OF THE INVENTION

### Definitions

“Cavity” refers to an unfilled space within a mass or substrate.

“Channel” refers to an enclosed passage between substrates or within a substrate.

“Microchannel” refers to an enclosed passage with micro-scale dimensions between substrates.

“Electrode” refers to a conductor used to establish electrical contact with a nonmetallic part of a circuit.

“Nanostructures” refers to structures having at least one nanoscale dimension such as nanotubes, nanorods, nanowires, nanoribbons, nanostripes, nanosheets, nanoropes, nanomeshes, nanohammocks, or thin film stacks comprised of a discrete number of thin films thinner than 100 nm in thickness each. An example of a nanostructure is a nanomesh of a nano material such as single or dual atomic layer carbon, graphene.

### Detailed Description

The present invention may be used to detect a variety of substances, including clusters of atoms (e.g., Hg, Au, and Pb), specific ions, chemical substances, biologically-related substances (e.g., molecules and macromolecules, such as proteins, nucleic acids, RNA and DNA), and whole biological cells. The sensor comprises one or more arrays of one or a plurality of nanostructures, which interact with atoms, chemical substances, and molecules in their surroundings. The affinity of these arrays of one or more nanostructures for specific target analytes and species is enhanced by the binding of high affinity and selective elements such as nucleic acids, aptamers, peptides, enzymes, antibodies, antibody fragments (e.g. minibodies, diabodies, cys-diabodies, Fab fragments and F(ab′)2 fragments), or a combination thereof onto the surface of the nanostructures. These high affinity and selective elements serve as links between nanostructures and analytes of interest such that their interaction can be enhanced, detected and quantified at large (mili-molar and micro-molar) and very low analyte concentrations (e.g., nano-, pico-, and femto-molar concentrations).

The sensor has the capability to separate and decouple microfluidic control and circulation from ionic, electrochemical, and/or electrostatic detection. The sensor has also the capability to separate microfluidic control and circulation from electrical inputs/outputs into the sensors. The microfluidics, for example, may be controlled from one side of the device; and the electronic and electrical input/outputs for detection can be controlled from the opposite side of the device.

The sensor may be used in a variety of applications. These applications include, but are not limited to the following: (a) disease detection, including early disease detection and screening; (b) diagnostics, (c) monitoring of analytes, disease indicators, and biomarkers for personalized therapeutics; and (d) measure molecular interactions. Other potential applications include analyte detection for water quality control, environmental monitoring of underground water resources and detection of underground contaminants, environmental monitoring of water reservoirs and sea water, monitoring of potable water for protection against biological and biochemical terrorism, and strategic monitoring of water resources for national security.

The present invention can be classified onto two main kinds of embodiments: open cavity embodiments and enclosed microchannel embodiments. First, the open cavity embodiments are described starting with the building-block components and elements that are critical to the invention. Next, the enclosed microchannel embodiments are described including the building-block components and elements that are critical to the present invention. Subsequently, utility and functional advantages of the present invention are described. This section ends with a description of the method of detection and analysis that is attainable with the present invention.

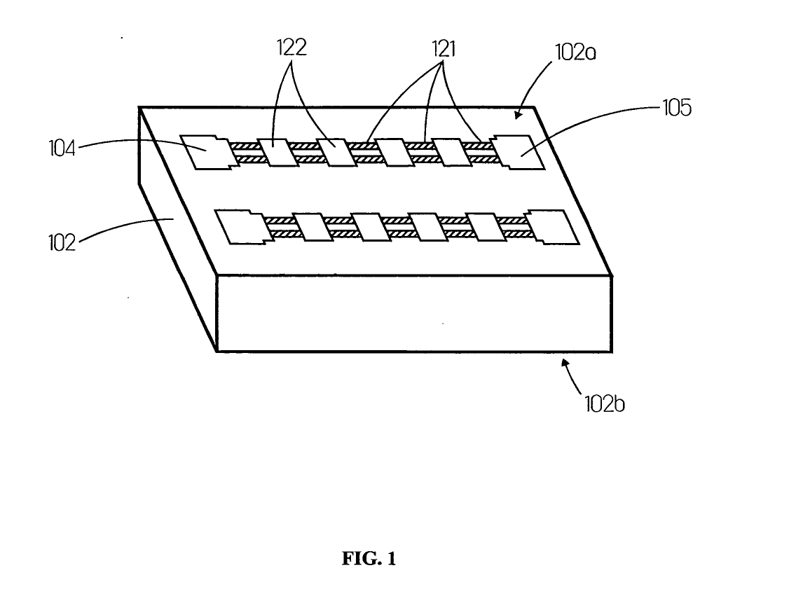

### Open-Cavity Embodiments: FIGS. 1-8

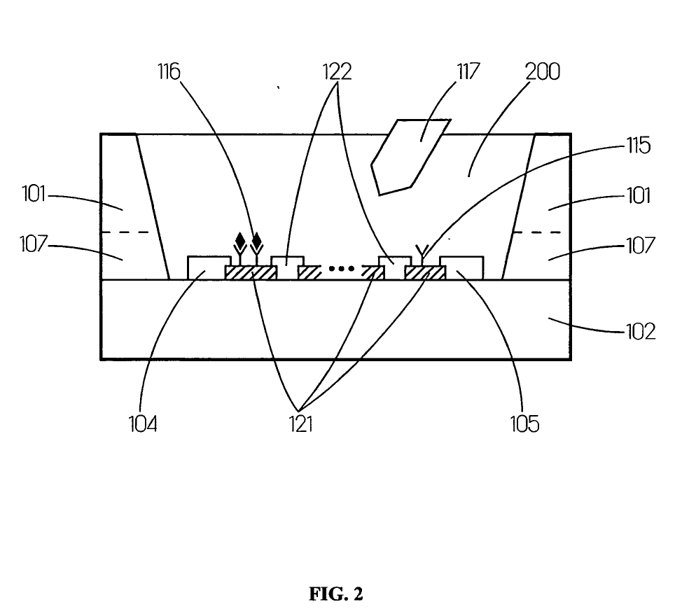

Elements of the present invention are described in FIG. 1, which shows arrays comprised of one or more nanostructures 121 on the front surface 102a of a layer or substrate 102. The arrays of one or more nanostructures 121 are electrically connected in parallel, in series, or a combination thereof at one end by a source electrode 104, in the middle by discrete electrical connectors 122, and at the second end by a drain electrode 105. The second component of the present invention is substrate 101. One embodiment of the present invention is described in reference to FIG. 2, where substrate 101 comprises a through-substrate cavity (TSC) 200. FIG. 2 shows a lateral cross-section diagram of substrates 101 and 102. In this embodiment, the sensor is comprised of two substrates that come together in “face-to-face” fashion. Substrate 101 comprises the TSC 200 and microchannels 107 that allow for the introduction and exit of a sample during analysis. Substrate 102 comprises the arrays of one or more nanostructures 121, source electrode 104, drain electrode 105, and discrete electrical connectors 122. The arrays of one or more nanostructures 121 are electrically connected in parallel, in series, or a combination thereof at one end by a source electrode 104, in the middle by discrete electrical connectors 122, and at the second end by a drain electrode 105. An external gate electrode probe 117 is inserted into the sensing cavity 200, also referred to as TSC, where the sample is introduced. During detection and analysis, target analytes 116 bind to high affinity species 115 on the surface of one or more nanostructures 121.

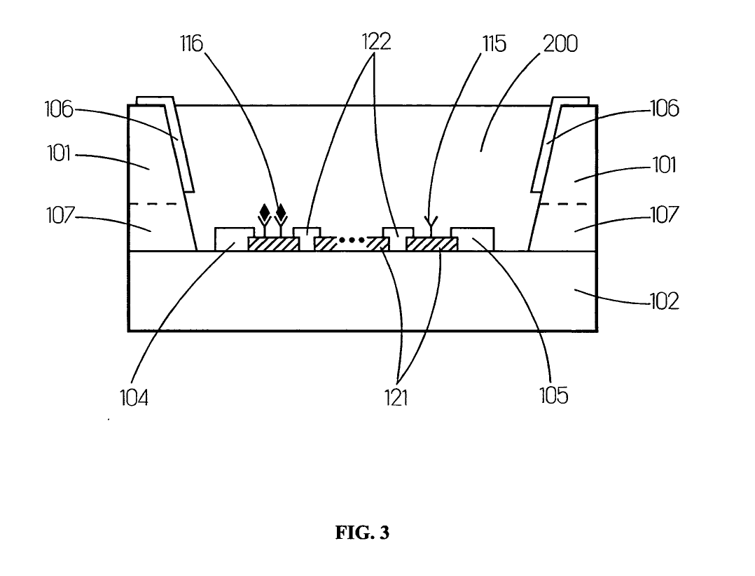

A slightly different embodiment of the present invention is described in reference to FIG. 3, which also shows a lateral cross-section diagram of substrates 101 and 102. In this embodiment, the sensor is also comprised of two substrates that come together in “face-to-face” fashion. Substrate 101 comprises the sensing TSC 200 and microchannels 107 that allow for the introduction and exit of a sample during analysis. No external gate electrode probe is used. Instead, a gate electrode 106 runs along the sidewall of the sensing TSC 200 and extends to the top surface of substrate 101. Substrate 102 comprises one ore more arrays of one or more nanostructures 121, source electrode 104, drain electrode 105, and discrete electrical connectors 122. The arrays of one or more nanostructures 121 are electrically connected in parallel, in series, or a combination thereof at one end by a source electrode 104, in the middle by discrete electrical connectors 122, and at the second end by a drain electrode 105. As displayed, target analytes 116 bind to high affinity species 115 on the surface of one or more nanostructures 121 during sensing and analysis.

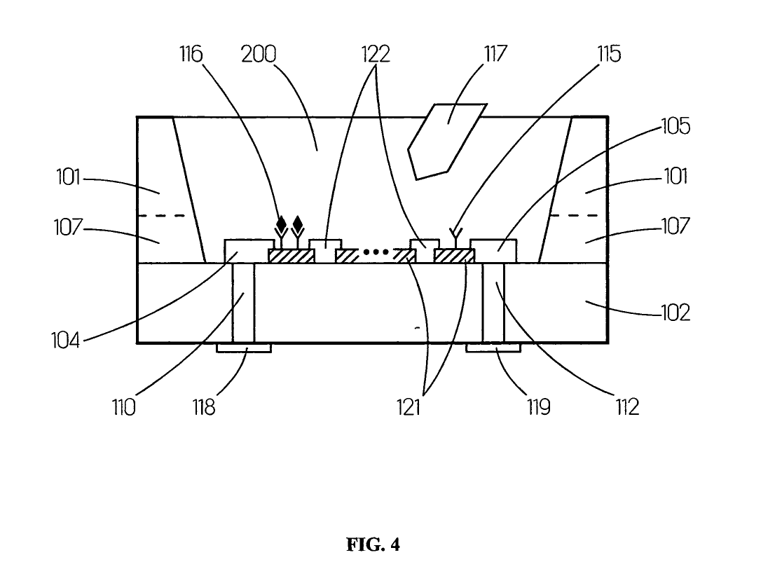

Another embodiment of the present invention is described in reference to FIG. 4, which also shows a lateral cross-section diagram of substrates 101 and 102. In this embodiment, the sensor is comprised of two substrates that come together in “face-to-face” fashion. Substrate 101 comprises the sensing TSC 200 and microchannels 107 that allow for the introduction and exit of a sample during analysis. Substrate 102 comprises arrays of one or more nanostructures 121, source electrode 104, drain electrode 105, discrete electrical connectors 122, and through-substrate vias (TSV) 110 and 112, which are connected to the source electrode 104 and drain electrode 105, respectively. The arrays of one or more nanostructures 121 are electrically connected in parallel, in series, or a combination thereof at one end by a source electrode 104, in the middle by discrete electrical connectors 122, and at the second end by a drain electrode 105. Metal traces 118 and 119 on the back surface of substrate 102 are connected to TSVs 110 and 112, respectively. These metal traces 118 and 119 are points of electrical connection to external power supply systems and/or devices. An external gate electrode probe 117 is inserted into the sensing cavity 200 for analysis and detection when target analytes 116 bind to high affinity species 115 on the surface of one or more nanostructures 121.

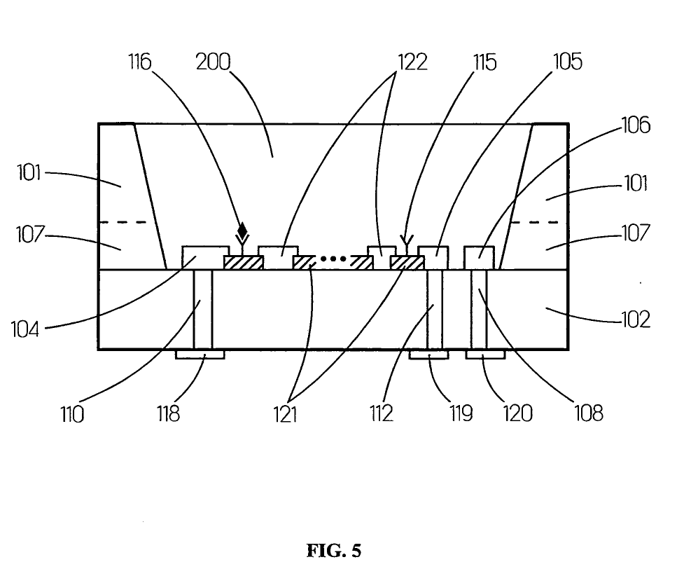

Another embodiment of the present invention is described in reference to FIG. 5, which also shows a lateral cross-section diagram. Similarly, the sensor is comprised of two substrates 101 and 102 that come together in “face-to-face” fashion. Substrate 101 comprises the sensing TSC 200 and microchannels 107 that allow for the introduction and exit of a sample during analysis. Substrate 102 comprises one or more arrays of one or more nanostructures 121, source electrode 104, drain electrode 105, discrete electrical connectors 122, and through-substrate vias (TSV) 110 and 112, which are connected to the source 104 and drain electrode 105 respectively. The arrays of one or more nanostructures 121 are electrically connected in parallel, in series, or a combination thereof at one end by a source electrode 104, in the middle by discrete electrical connectors 122, and at the second end by a drain electrode 105. Metal traces 118 and 119 on the back surface of substrate 102 are connected to TSVs 110 and 112, respectively. In this embodiment there is no external gate probe, but there is a gate electrode 106 on the front surface of substrate 102. Gate electrode 106 is connected to TSV 108, which is connected to metal trace 120 on the back surface of substrate 102. Therefore, metal traces 118, 119, and 120 on the back surface of substrate 102 are electrically connected to electrodes 104, 105, and 106, respectively. Target analytes 116 bind to high affinity species 115 on the surface of one or more nanostructures 121 during sensing and analysis.

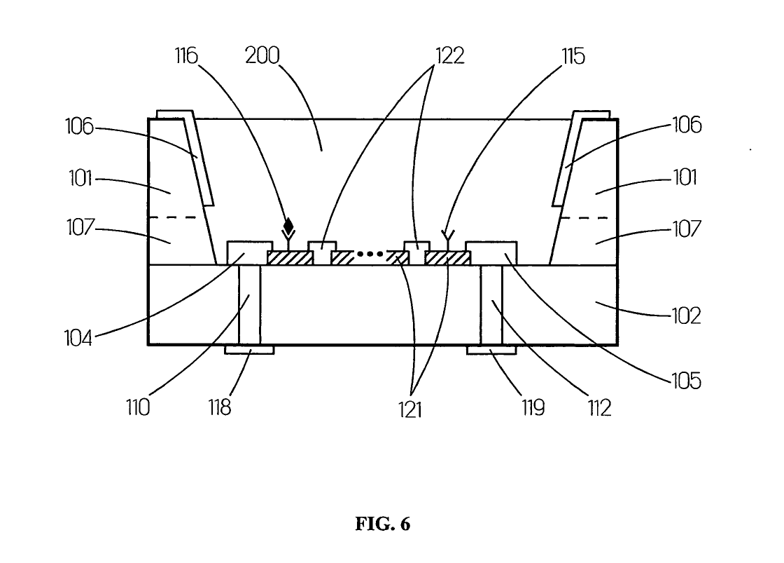

An alternative embodiment of the present invention is described in reference to FIG. 6. Instead of utilizing an external gate probe, this embodiment comprises a gate electrode 106 that runs along the sidewall of sensing TSC 200 and extends to the top surface of substrate 101. Substrate 101 comprises the sensing TSC 200 and microchannels 107 that allow for the introduction and exit of a sample during analysis. Substrate 102 comprises one or more arrays of one or more nanostructures 121, source electrode 104, drain electrode 105, and discrete electrical connectors 122. The arrays of one or more nanostructures 121 are electrically connected in parallel, in series, or a combination thereof at one end by a source electrode 104, in the middle by discrete electrical connectors 122, and at the second end by a drain electrode 105. Source electrode 104 is electrically connected to metal trace 118 via TSV 110. Similarly, drain electrode 105 is electrically connected to metal trace 119 via TSV 112. During sensing and analysis, target analytes 116 bind to high affinity species 115 on the surface of one or more nanostructure 121.

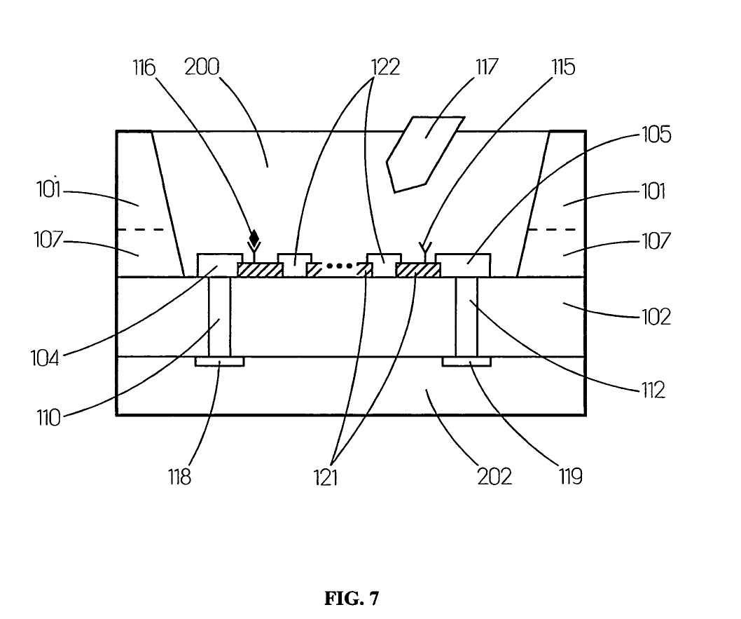

FIG. 7 displays an embodiment of the present invention that is similar to the embodiment described in FIG. 4. In addition to comprising all the different elements described in FIG. 4, this embodiment further comprises an integrated circuit 202, which is attached to the back surface of substrate 102. The connection of the integrated circuit 202 to the metal traces 118, 119, and 120 enables additional miniaturization of the sensor since electrical inputs and outputs can be programmed, controlled, and recorded by the integrated circuit 202 during sensing and analysis.

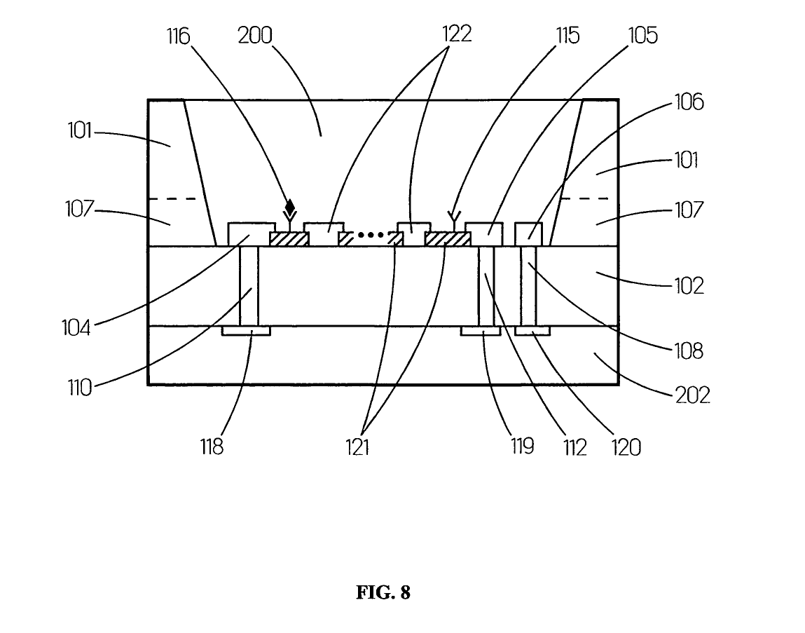

FIG. 8 displays an embodiment of the present invention that is similar to the embodiment described in FIG. 5, where a gate electrode 106 is located on the front surface of substrate 102, and said gate electrode 106 is electrically connected to metal trace 120 via TSV 108. In addition to comprising all the different elements described in FIG. 5, this embodiment further comprises an integrated circuit 202, which is attached to the back surface of substrate 102. The connection of the integrated circuit 202 to the metal traces 118, 119, and 120 enables additional miniaturization of the sensor because electrical inputs and outputs can be programmed, controlled, and recorded by the integrated circuit 202 during sensing and analysis.

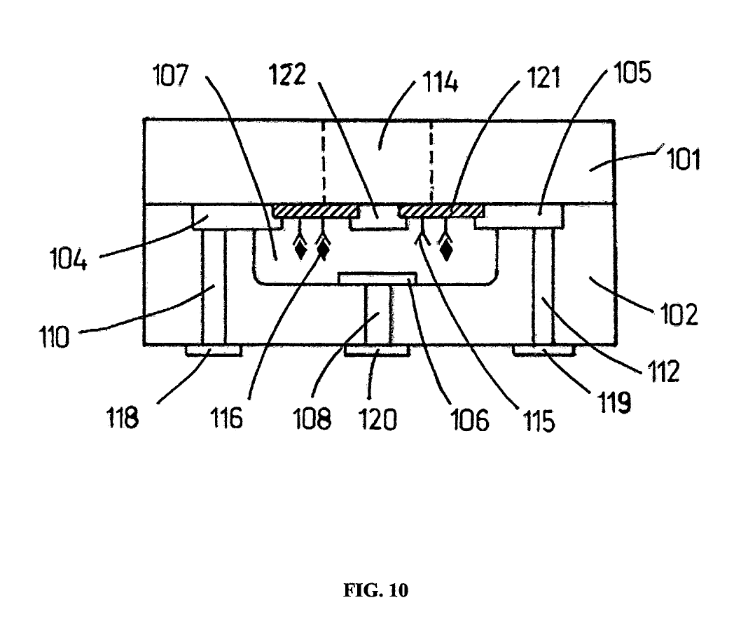

### Enclosed Microchannel Embodiments: FIGS. 10-19

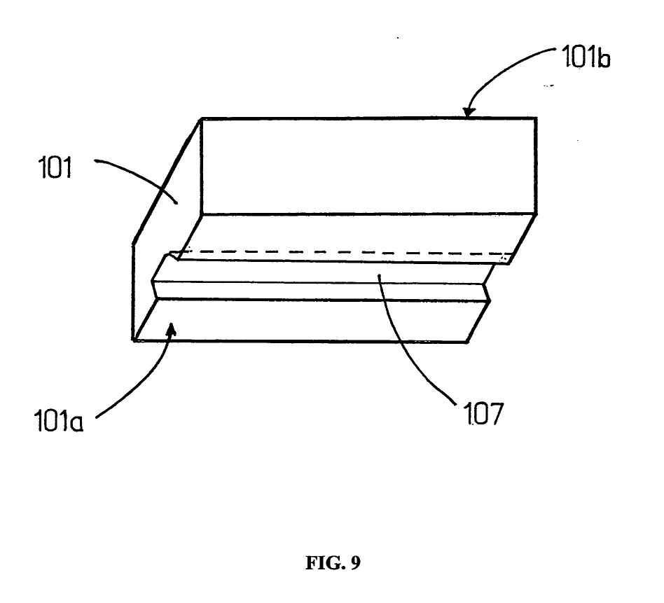

Elements for this family of embodiments are described in FIG. 1, which displays one or more arrays of one or more nanostructures 121, one or more discrete electrical connectors 122, a source electrode 104, and a drain electrode 105 on a layer or substrate 102. Another critical element is described with reference to FIG. 9, which shows a microchannel 107 on the bottom surface 101a of a layer or substrate 101. In reference to FIG. 1, the one or more arrays of one or more nanostructures 121 are electrically connected in parallel, in series, or a combination thereof at one end by a source electrode 104, in the middle by discrete electrical connectors 122, and at the second end by a drain electrode 105. In reference to FIG. 9, substrate 101 comprises one or a plurality of horizontal microchannels 107, which on a few possible embodiments are connected to a plurality of vertical channels. The embodiments described in this section have at least two substrates 101 and 102, which come together in “face-to-face” fashion. Substrate 101 has a front surface 101a and a back surface 101b (FIG. 9), and substrate 102 has a front surface 102a and a back surface 102b (FIG. 1). The enclosed channel embodiments are subdivided into two groups: Embodiments that have the one or more arrays of one or more nanostructures 121 on surface 101a and microchannels 107 on surface 102a, and embodiments that have the one or more arrays of one or more nanostructures 121 on surface 102a and microchannels 107 on surface 101a.

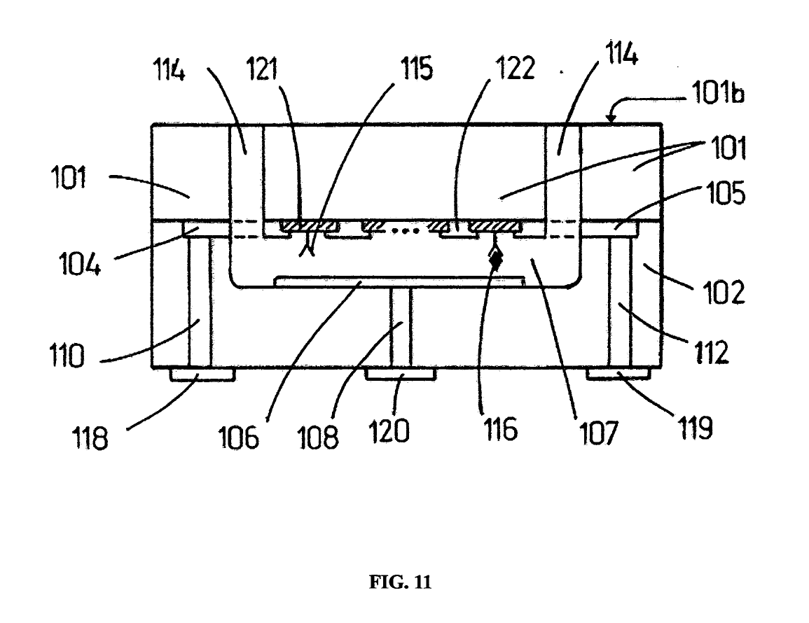

One embodiment of the present invention is described in reference to FIG. 10, which shows a side view cross-section diagram of substrates 101 and 102. Substrate 101 comprises vertical channels 114, one ore more arrays of one or a plurality of nanostructures 121 electrically connected in parallel, in series, or a combination thereof by a source electrode 104 at one end, by discrete electrical connectors 122 in the middle, and by a drain electrode 105 at the second end. Vertical channels 114 allow for the introduction and exit of a sample to microchannel 107 on substrate 102 during sample analysis. Substrate 102 comprises microchannel 107, gate electrode 106, TSV 108, and metal trace 120. Source electrode 104 is connected to metal trace 118 via TSV 110. Similarly, drain electrode 105 is connected to metal trace 119 via TSV 112. Target analytes 116 bind to high affinity species 115 on the surface of one or more nanostructures 121 during sample sensing and analysis. A different side view cross-section of this embodiment is displayed in FIG. 11.

In reference to FIG. 11, an array of one or more nanostructures 121 are present on the front surface of substrate 101; microchannels 107 are etched or mechanically formed on the front surface of substrate 102. Electrically conductive through-layer means, which are also referred to as through-substrate vias (TSVs), are included in substrate 102. These through-layer conductive vias are the shortest path of electrical connection between the front side and the back side of substrate 102. Gate electrode 106 runs along microchannel 107 on the front surface of substrate 102. This integrates source electrode 104, the one or more arrays of one or more nanostructures 121, gate electrode 106, discrete electrical connectors 122, and drain electrode 105 into one or a plurality of functional nanostructured-array field-effect-transistors (NSA-FETs), which can be operated and controlled from the back surface of substrate 102 when using an external power supply and an integrated circuit/system. These embodiments also comprise channels with reservoirs and pillars for the mechanical separation of different biological substances in a sample prior to analyte detection although these elements cannot be viewed in the perspectives displayed in FIG. 10 and FIG. 11.

Vertical channels 114 connect both sides of substrate 101 such that a fluid or gas sample can flow from back side 101b into sensing microchannel 107, then through a second set of vertical channels 114 back to surface 101b to exit the device. In this embodiment, microfluidic control is conducted from surface 101b. The electronic current/voltage (“I/V”) characteristics are controlled from back side 102b using an external integrated circuit and power supply.

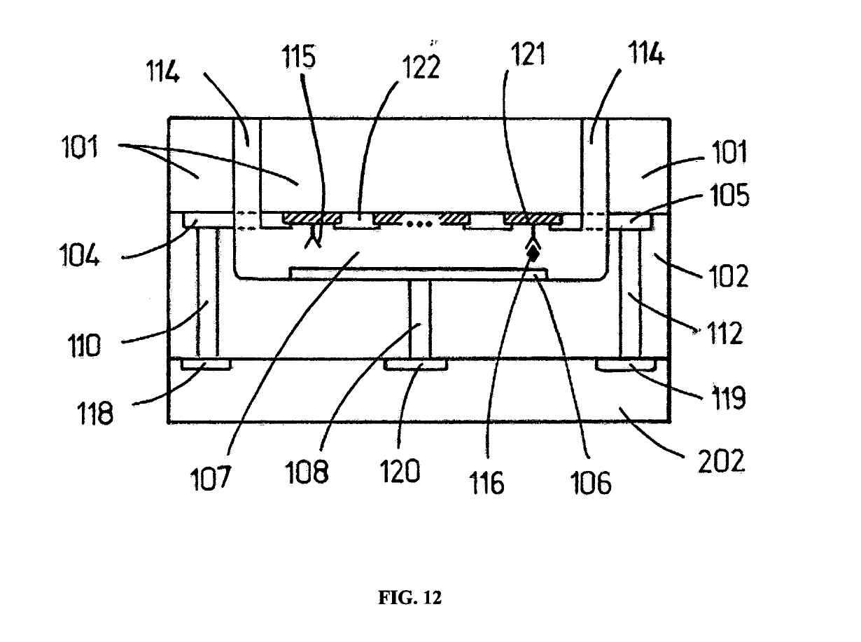

FIG. 12 displays an embodiment of the present invention that is similar to the embodiment described in FIG. 11. In addition to comprising all the different elements described for the previous embodiment in FIG. 11, this embodiment comprises an integrated circuit 202, which is attached to the back surface of substrate 102. The integrated circuit 202 is connected to the metal traces 118, 119, and 120. This enables additional miniaturization of the sensor since electrical inputs and outputs can be programmed, controlled, and recorded by the integrated circuit 202. This embodiment also comprises channels with reservoirs and pillars for the mechanical separation of different biological substances in a sample prior to analyte detection although these elements cannot be viewed in the perspective displayed in FIG. 12.

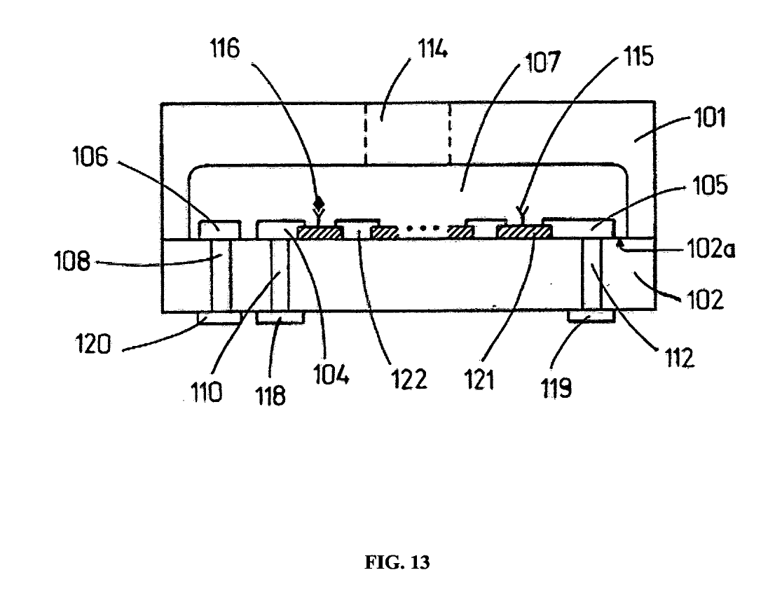

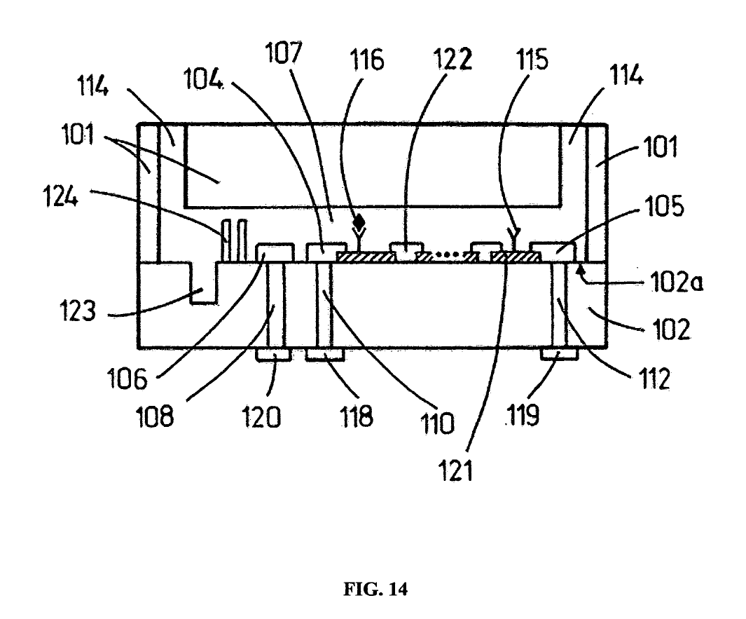

Having the one or more arrays of one or a plurality of nanostructures on surface 102a (FIG. 1) and microchannel 107 on surface 101a (FIG. 9) gives rise to multiple embodiments. One embodiment of the present invention with this characteristic is described in reference to FIG. 13, which shows a lateral cross-section diagram. Similarly, the sensor is comprised of two substrates 101 and 102 that come together in “face-to-face” fashion. Substrate 102 comprises one or more arrays of one or a plurality of nanostructures 121, a source electrode 104, discrete electrical connectors 122, a drain electrode 105, and gate electrode 106 on surface 102a. The arrays of one or more nanostructures 121 are electrically connected in parallel, in series, or a combination thereof at one end by a source electrode 104, in the middle by discrete electrical connectors 122, and at the second end by a drain electrode 105. The electrodes 104, 105, and 106, are connected to metal traces 118, 119, and 120 via TSVs 110, 112, and 108, respectively. Substrate 101 comprises microchannels 107 and vertical channels 114 for the introduction and exit of a sample during detection and analysis. This embodiment also comprises channels with reservoirs and pillars for the mechanical separation of different biological substances in a sample prior to analyte detection although these elements cannot be viewed in the perspective displayed in FIG. 13. Target analytes 116 bind to high affinity species 115 onto the surface of one or more nanostructures 121 during sensing and analysis. This embodiment is also displayed in FIG. 14, but the view corresponds to an orthogonal side view cross-section where an array of one or more nanostructures 121 are visible on surface 102a. All the elements described in FIG. 13 are also present in this figure. The embodiment displayed in FIG. 14 comprises reservoirs 123 and pillars 124 for the mechanical separation of different biological substances in a sample prior to analyte detection.

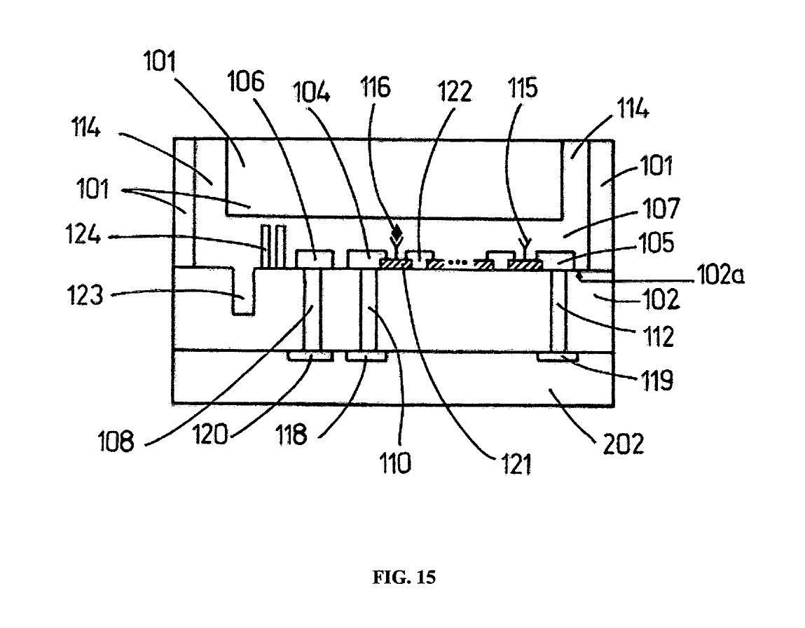

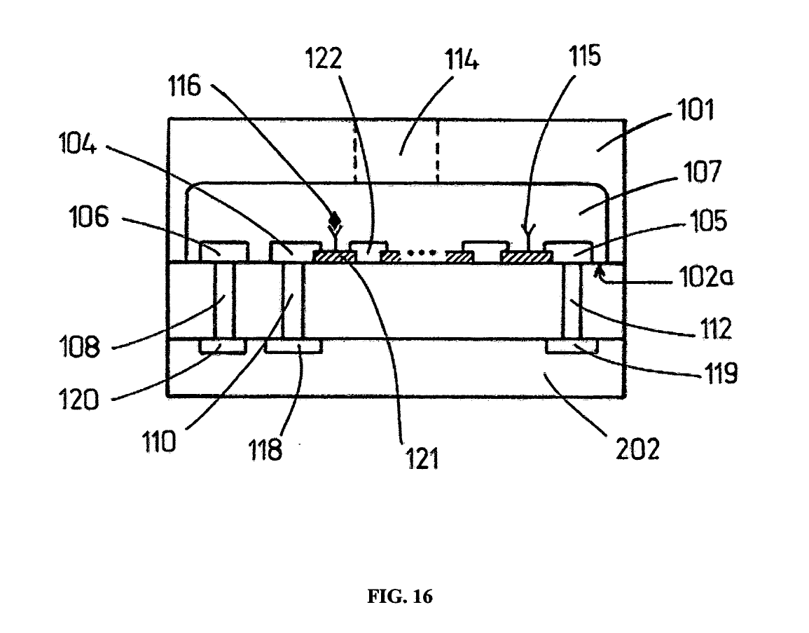

A similar embodiment is displayed with reference to FIG. 15. In addition to all the elements described in FIG. 13 and FIG. 14, this embodiment further comprises an integrated circuit 202 connected to the back surface of substrate 102. The integrated circuit 202 is connected to the metal traces 118, 119, and 120, which enables additional miniaturization of the sensor since electrical inputs and outputs can be programmed, controlled, and recorded by the integrated circuit 202. This embodiment is displayed in FIG. 16 from a different perspective. An orthogonal side view is displayed to show how target analytes 116 bind to high affinity species 115 on the surface of one or more nanostructures 121 during sensing and sample analysis.

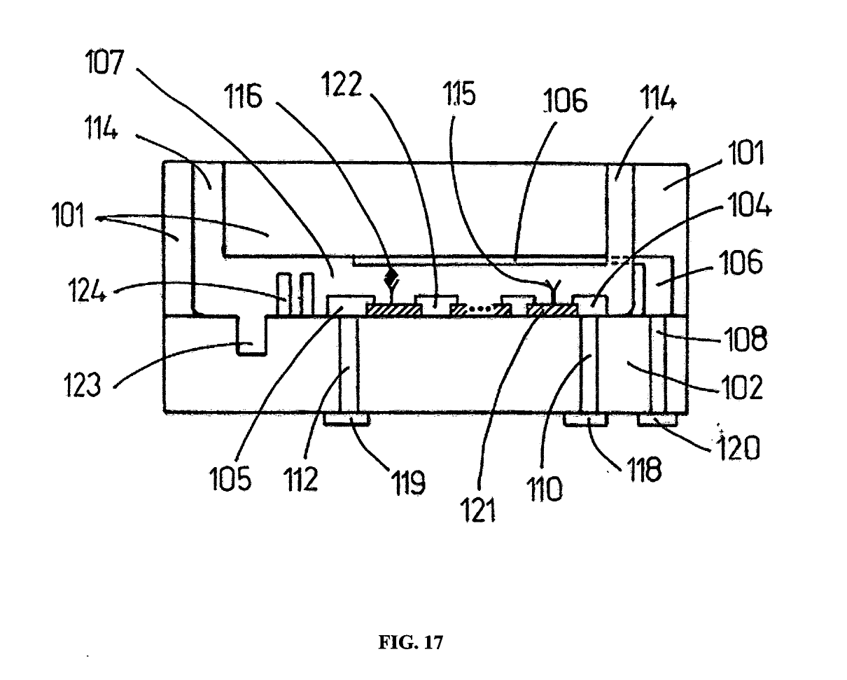

A slightly different embodiment is described with reference to FIG. 17. In this embodiment, the one or more arrays of one or more nanostructures 121 are horizontally placed on surface 102a and electrically connected in parallel, in series, or a combination thereof by discrete electrical connectors 122, and microchannels 107 are etched or mechanically formed on the front surface of substrate 101. Gate electrode 106 runs along microchannel 107 and is connected to metal trace 120 via TSVs 108. Source electrode 104 on surface 102a is placed between the one or more arrays of one or more nanostructures 121 and TSV 110, which is connected to metal trace 118. Similarly, drain electrode 105 is placed between the one or more arrays of one or more nanostructures 121 and TSV 112, which is connected to metal trace 119. This embodiment integrates source electrode 104, the one or more arrays of one or more nanostructures 121, discrete electrical connectors 122, gate electrode 106, and drain electrode 105 into one or a plurality of functional NAS-FETs. Substrate 101 comprises microchannels 107, which are connected to vertical channels 114 to enable the introduction and exit of samples for sensing and analysis. The embodiment displayed in FIG. 17 comprises reservoirs 123 and pillars 124 for the mechanical separation of different biological substances in a sample prior to analyte detection.

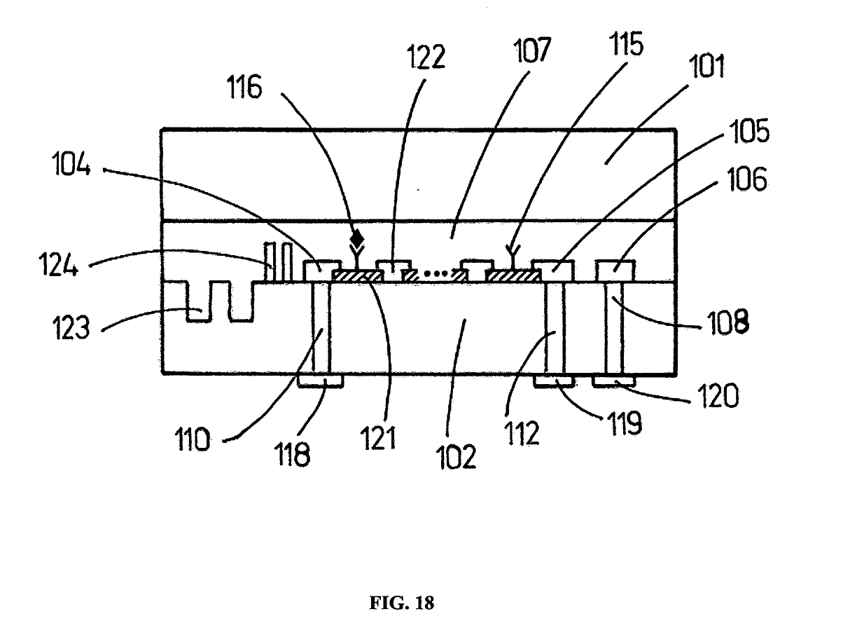

A different embodiment is described with reference to FIG. 18. In this embodiment, one or more arrays of one or a plurality of nanostructures 121 are placed on substrate 102 and these are electrically connected in parallel, in series, or a combination thereof by a source electrode 104 at one end, by discrete electrical connectors 122 in the middle, and by a drain electrode 105 at the second end. Source electrode 104 is electrically connected to metal trace 118 via TSV 110. Drain electrode 105 is electrically connected to metal trace 119 via TSV 112. Gate electrode 106 is located on the front surface of substrate 102, and it is electrically connected to metal trace 120 via TSV 108. Microchannel 107 is formed on the front surface of substrate 101, and said microchannel 107 runs along the width of the device as described in FIG. 18. Microchannel 107 provides for the introduction and exit of a sample during analysis, and reservoirs 123 and pillars 124 enable the mechanical separation of different biological substances in a sample prior to analyte detection. Target analytes 116 bind to high affinity species 115 on the surface of one or more nanostructures 121 during sensing and sample analysis.

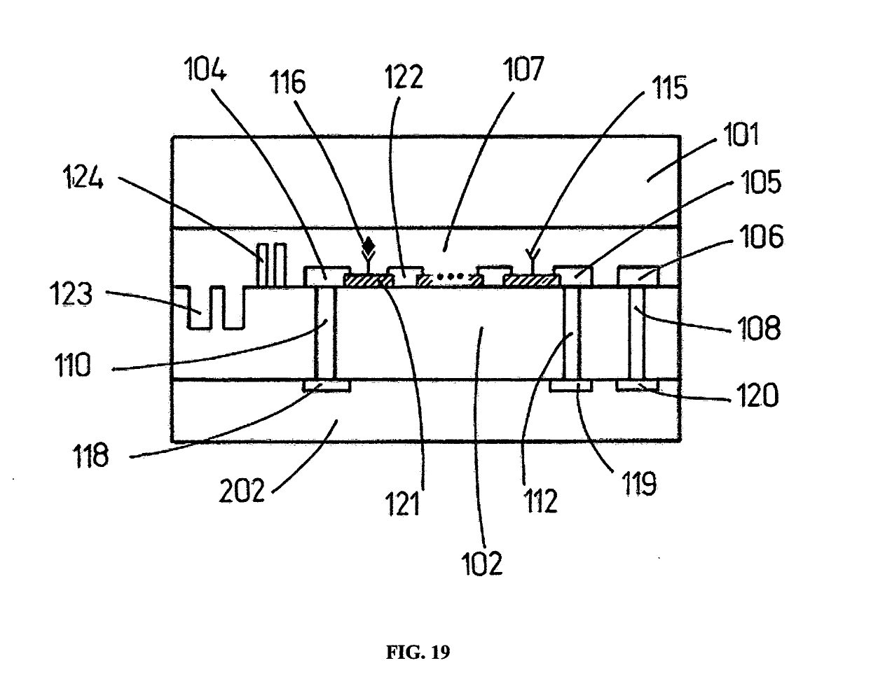

A slightly different embodiment is described with reference to FIG. 19. In addition to including all the elements described in FIG. 18, this embodiment comprises an integrated circuit 202 attached to the back surface of substrate 102. Said integrated circuit 202 is electrically connected to metal traces 118, 119, and 120, and can consequently control the electrical inputs and record electrical outputs of the NAS-FET formed by the electrodes 104, 105, 106, discrete electrical connectors 122, and the one or more arrays of one or more nanostructures 121.

### Utility and Functional Advantages

Since the microfluidic and the electronic controls are decoupled to the opposite sides of the device as described in figures FIG. 4-8 and FIG. 10-19, other complementary operations can be added to the device. For instance, if substrates 101 and 102 are transparent, or translucent to light, and a light source (e.g., laser, UV, infrared, or visible) is illuminated from one side of the device, then fluorescence light and/or optical output can be collected and measured from the opposite side of the device for the case of the embodiments described in FIG. 4-6, FIG. 10-11, FIG. 13-14, and FIG. 17-18, which are embodiments that do not comprise an integrated circuit 202. If only one substrate is transparent or translucent, substrate 101 or 102, and the other substrate reflects light (e.g., laser, UV, IR or visible), then a light source and an output detector can be placed on the same side of the present invention. Consequently, fluorescence light and/or optical output can be collected and measured. These utility advantages are particularly relevant with respect to the embodiments described in FIG. 4-8 and FIG. 10-19. Using an external light source (e.g., laser, UV, IR or visible), the light is used to trigger photochemical-interactions between the high affinity species (e.g., nucleic acids, aptamers, antibodies, or antibody fragments) on the surface of one or more nanostructures 121 with the analyte species of interest contained in the sample. These photochemical-interactions facilitate complementary forms of molecular characterization using optical means (e.g., laser, optical fluorescence, fluorescence resonance energy transfer (FRET), or other).

### Molecular Detection, Sensing, and Analysis Method

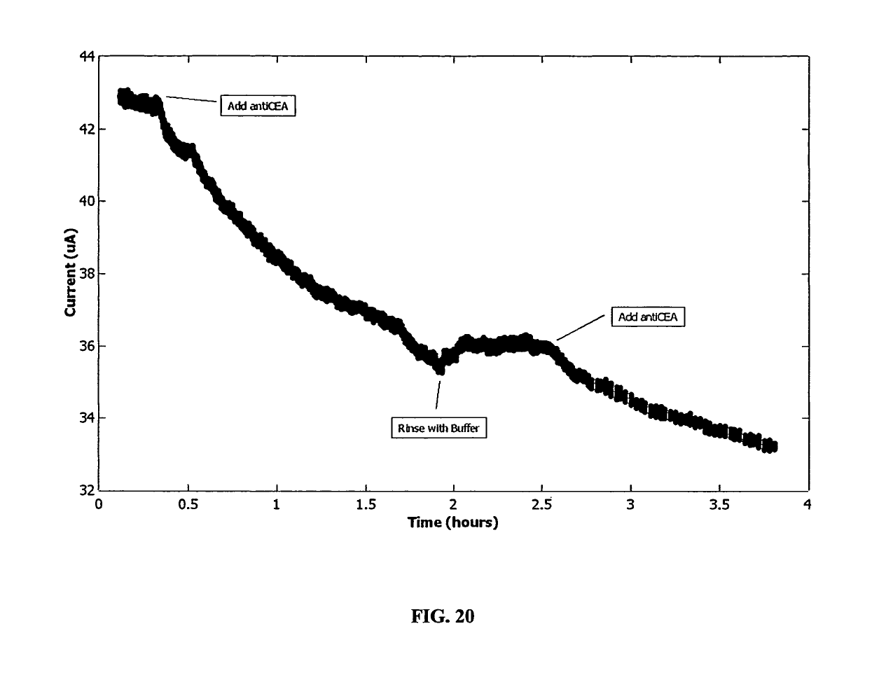

A method according to the present invention is described in relation to FIG. 2-8, FIG. 10, FIG. 13, and FIG. 17. A solution of known concentration containing nucleic acids, antibodies, antibody fragments, peptides, DNA, RNA, enzymes, or engineered antibody fragments, or a combination thereof is introduced into sensing cavity 200 or microchannels 107 to coat, functionalize, and add target affinity to the one or more arrays of one or a plurality of nanostructures 121. Nucleic acid molecules (e.g. aptamers), antibody molecules, antibody fragments, peptides, DNA, RNA, enzymes, or engineered antibody fragments 115 bind to the surface of one of more nanostructures 121. This step is displayed in FIG. 20 where carcino-embryonic antigen (CEA) antibody fragments, Anti-CEA in a 10 μg/ml, are introduced into the sensor to functionalize the surface of a nanostructure 121, which in this case is a nanosheet of graphene. Changes in surface potential are detected in the form of changes in current (μA) as the Anti-CEA fragments (proteins) immobilize onto the surface of the nanosheet.

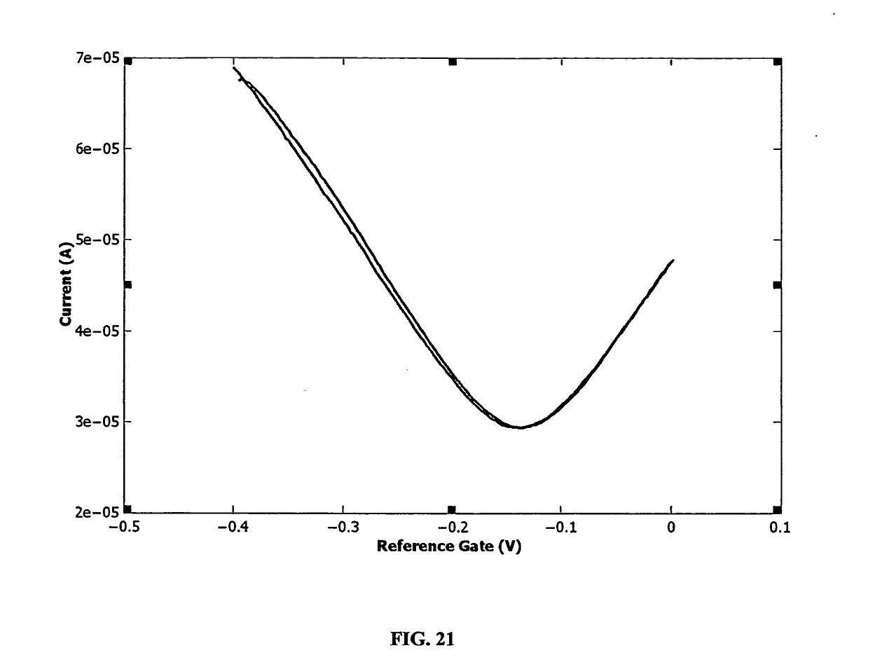

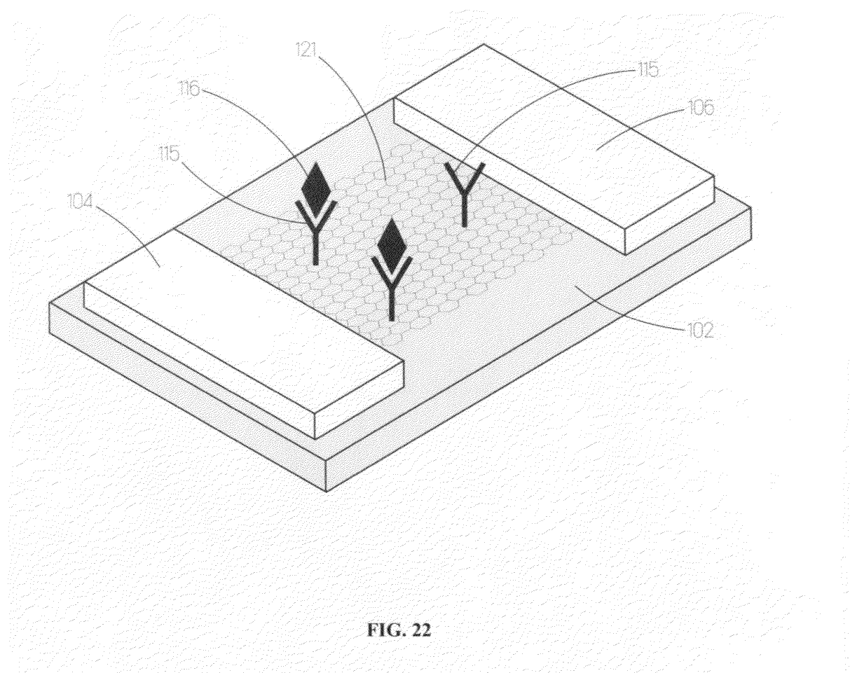

A buffer electrolyte solution is introduced into the sensing cavity 200 or microchannels 107. The electrolyte solution permits the activation of the NAS-FETs at their baseline current/voltage (I/V), which defines a reference state and it is equivalent to zero concentration of the measured targeted specie or analyte (e.g., protein biomarkers). This step is executed as part of the calibration procedure of the present invention. This step is displayed in FIG. 21 where a baseline curve with a normal V-shape demonstrates utility of a NAS-FET, using a graphene nanosheet 121, to sense and detect changes in the surface potential of the integrated sensor as a function of the reference gate potential in solution. Subsequently, in order to complete the calibration, a solution containing a reagent or analyte of known concentration is mixed with the buffer solution and introduced into the device. The analyte species of known concentration 116 bind to the high affinity and selectivity species 115 on the surface of one or more nanostructures 121 to collect an standardized I/V electrical measurement. This will cause the V-shape curve to shift to the right or left away from its initial baseline state. The amount of shift will be set directly proportional to the sample concentration. An integrated sensor comprised of a graphene nanosheet 121 is described in FIG. 22.

Finally, a known quantity of a sample (e.g., known quantity of blood, plasma serum, or biological fluid) is mixed with known quantities of an electrolyte solution and/or reagents in order to be introduced into the sensing cavity 200 or the sensing microchannel 107 through channels 114. For all embodiments described in FIGS. 10-18, reservoirs 123 and pillars 124 enable the mechanical separation and filtration of different biological substances in a sample prior to analyte detection in microchannel 107. For example, reservoirs 123 and pillars 124 enable the device to separate biological cells from serum in blood samples. This simplifies and grants in-situ sample preparation capabilities to the sensor device.

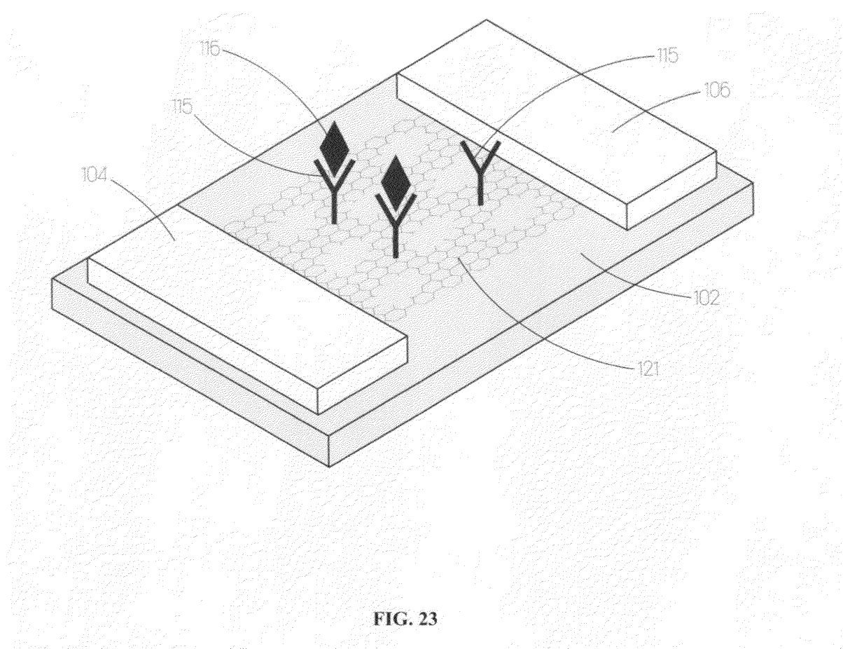

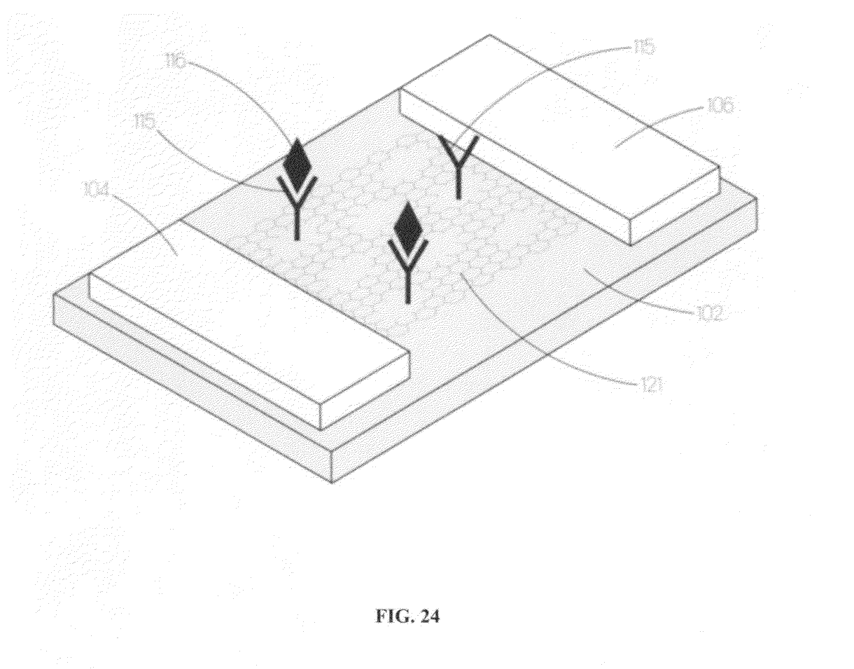

Detection and sensing measurements can be performed with other similar embodiments of this integrated sensor. For example, FIG. 23 describes how target ligands 116 bind to affinity receptors 115 on the surface of a graphene nanomesh (nanostructure) 121 during sensing and sample analysis. Similarly, FIG. 24 describes how target ligands 116 bind to affinity receptors 115 on the surface of a substrate 102 within the holes of a graphene nanomesh (nanostructure) 121 during sensing and sample analysis.

The arrays of NAS-FETs at the bottom of the sensing cavity 200 or inside each sensing microchannel 107 serve as signal amplifiers and enable the detection and measurement of changes in I/V characteristics caused by the binding events between high affinity ligands 115 and targeted analytes 116 (e.g., protein biomarkers or nucleic acids) on the surface of one or more nanostructures 121. For example, in a blood serum analysis, the recorded I/V characteristics for a specific ligand-analyte pair 115-116 on the surface of one or more nanostructures 121 will be directly correlated to the concentration and/or quantification of said analyte 116 in the sample. The compilation of measurements of multiple types of analytes 116 defines a signature-analyte-profile (SAP) or signature-protein-profile, which is unique to each individual sample (e.g., blood serum sample).

Sensing cavity 200 or microchannels 107 may be cleaned and reused. This is done by flushing the sensing cavity 200 or microchannels 107 with a cleaning solution and re-functionalizing the one or more arrays of one or more nanostructures 121 with a new set of high affinity and selective species 115. A subsequent analysis with the same or different set of target analytes 116 (e.g., proteins or nucleic acids) is performed to gather more information for the signature-analyte-profile (SAP).

### List of Elements

The following is a list of elements comprised in the present invention.

| Number | Element |
|---|---|
| 101 | First layer or substrate |
| 101a | front surface of substrate 101 |
| 101b | back surface of substrate 101 |
| 102 | Second layer or substrate |
| 102a | front surface of substrate 102 |
| 102b | back surface of substrate 102 |
| 104 | source electrode |
| 105 | drain electrode |
| 106 | gate electrode |
| 107 | microchannel or microchannels, also referred to as sensing |
| | microchannel |
| 108 | gate TSV, where through-substrate via (TSV) |
| 110 | source TSV |
| 112 | drain TSV |
| 114 | vertical channel or channels |
| 115 | high affinity and selectivity species, receptors |
| 116 | target analytes, ligands |
| 117 | external gate electrode probe |
| 118 | source metal trace |
| 119 | drain metal trace |
| 120 | gate metal trace |
| 121 | nanostructure, or array of one or a plurality of nanostructures |
| | (e.g. graphene nanosheet, graphene nanomesh) |
| 122 | discrete electrical connectors |
| 123 | reservoirs for mechanical separation of substances |
| 124 | pillars |
| 200 | through-substrate cavity (TSC), also referred to as sensing TSC |
| | cavity |
| 202 | integrated circuit |

1. A multilayer device for sensing ions, protons, non-biological molecules, biological molecules, or whole cells, wherein the device comprises:
a) one or more cavities that provide for the introduction of a sample to be analyzed and one or more channels that provide for exit of the sample, or one or more channels that provide for the introduction and exit of the sample;
b) one or more nanostructures presented to the one or more cavities or one or more channels;
c) a plurality of conductive elements electrically connected to the one or more nanostructures; and,
d) one or more gate electrodes presented to the one or more cavities or one or more channels.

2. The device according to claim 1, wherein the one ore more nanostructures are composed of a monolayer of carbon atoms, so-called graphene, with or without chemical doping, and positioned either in parallel or in series with one another or a combination thereof while being electrically connected to the conductive elements.

3. The device according to claim 1, wherein the one or more conductive elements are placed on an insulating layer.

4. The device according to claim 1, wherein one or more conductive elements are passivated with one or more layers of insulating and/or biologically repellent materials.

5. The device according to claim 1, wherein the one or more nanostructures are at least 1 micron long and these are passivated with one or more discrete layers of chemical binding elements applied to promote affinity for specific analytes or species: ions, protons, non-biological molecules, biological molecules, or whole cells.

6. The device according to claim 1, wherein the one or more gate electrodes are composed of a metal or a metallic alloy, and said electrodes are located on a channel wall opposite or adjacent to that of the nanostructures

7. The device according to claim 1, wherein the one of more nanostructures are suspended above or supported by a continuous layer such that one or more arrays of nanowires, nanoribbons, nanomeshes, nanosheets, super-lattices, nanotubes, nanohammocks, nanostripes, or nanorods are formed and connected to the one or more conductive elements.

8. The device according to claim 1, wherein the device further comprises a plurality of through layer conductive elements which provide short paths for electrical conduction.

9. The device according to claim 7, wherein the device comprises a first layer and a second layer, and wherein the first layer comprises a sensing cavity and microchannels allowing for the introduction and exit of the sample, and wherein the second layer comprises the one or more nanostructures, one or more source conductive elements, one or more drain conductive elements, one or more intermediate conductive elements, and wherein the gate electrode is external to the device.

10. The device according to claim 7, wherein the device comprises a first layer and a second layer, and wherein the first layer comprises a sensing cavity and microchannels allowing for the introduction and exit of the sample, and wherein the gate electrode runs along the sidewall of the sensing cavity and extends to the top of the first layer, and wherein the second layer comprises the one or more nanostructures, one or more source conductive elements, and one or more drain conductive elements.

11. The device according to claim 7, wherein the device comprises a first layer and a second layer, and wherein the first layer comprises a sensing cavity and microchannels allowing for the introduction and exit of the sample, and wherein the second layer comprises the one or more nanostructures, one or more source conductive elements, one or more drain conductive elements, and a plurality of through layer conductive elements, which provide short paths for electrical conduction, and wherein the one or more source conductive elements are connected to one or more first through layer conductive elements and the one ore more drain conductive elements are connected to one or more second through layer conductive elements, and wherein the gate electrode is external to the device.

12. The device according to claim 7, wherein the device comprises a first layer and a second layer, and wherein the first layer comprises a sensing cavity and microchannels allowing for the introduction and exit of the sample, and wherein the second layer comprises the one or more nanostructures, one or more source conductive elements, one or more drain conductive elements, and a plurality of through layer conductive elements which provide short paths for electrical conduction, wherein the one or more source conductive elements are connected to one or more first through layer conductive elements and the one ore more drain conductive elements are connected to one or more second through layer conductive elements, and wherein the gate electrode is included on an internal surface of the second layer such that it projects into the sensing cavity, and wherein the gate electrode is connected to a third through layer conductive element.

13. The device according to claim 7, wherein the device comprises a first layer and a second layer, and wherein the first layer comprises a sensing cavity and microchannels allowing for the introduction and exit of the sample, and wherein the gate electrode runs along the sidewall of the sensing cavity and extends to the top of the first layer, and wherein the second layer comprises the one or more nanostructures, one ore more source conductive elements, and one or more drain conductive elements, and wherein the one or more source conductive elements are connected to one or more first through layer conductive elements and the one ore more drain conductive elements are connected to one or more second through layer conductive elements.

14. The device according to claim 12, wherein the device further comprises a third layer, and wherein the third layer is an integrated circuit attached to the external surface of the second layer, and wherein the integrated circuit is connected to the first, second and third through layer conductive elements in the second layer.

15. The device according to claim 13, wherein the device further comprises a third layer, and wherein the third layer is an integrated circuit attached to the external surface of the second layer, and wherein the integrated circuit is connected to the first and second through layer conductive elements in the second layer, and wherein the gate electrode is connected to an external gate extension.

16. A method for sensing species such as ions, metals, one or more non-biological molecules, one or more biological molecules, and whole cells wherein the method comprises the steps of:
a) bringing into physical contact a solution of high affinity and selective binding elements with the device according to claim 1, wherein the high affinity and selective binding elements add functionality to the one or more nanostructures by binding species of interest to the surface of the nanostructures;
b) introducing a buffer-electrolyte solution into one or more cavities, or the one or more channels of the device, thereby allowing activation of the device for calibration purposes and for setting a baseline current or voltage reference state;
c) introducing a sample in gas or in solution into the one or more cavities, or one or more channels of the device and determining any changes in the current or voltage relative to the baseline state;
wherein the changes are correlated with the binding of one or more species of interest in the sample to the affinity binding elements on one or more nanostructures.

17. The method according to claim 16, wherein high affinity and selectivity binding elements are selected from a group of elements consisting of nucleic acid molecules, aptamers, peptides, enzymes, monoclonal antibodies, polyclonal antibodies, minibodies, diabodies, cys-diabodies, derived antibody fragments, or fab fragments.

18. The method according to claim 16, wherein the buffer-electrolyte solution promotes ionic exchange and transport.

19. The method according to claim 16, wherein molecular interactions can be measured as a function of changes in current, voltage, or impedance.
